(19) United States
(12) Patent Application Publication
(10) Pub. No.: US 2020/0216875 A1
(43) Pub. Date: Jul. 9, 2020

(54) NUCLEIC ACID DETERMINATION METHOD

(71) Applicant: ZHUHAI ASTROBIO BIOTECHNOLOGY CO, LTD., Zhuhai (CN)

(72) Inventors: Xing SU, Zhuhai (CN); Kaiyuan WU, Zhuhai (CN)

(21) Appl. No.: 16/648,317

(22) PCT Filed: Sep. 14, 2018
(86) PCT No.: PCT/CN2018/105634

§ 371 Date: Mar. 18, 2020

(30) Foreign Application Priority Data
Sep. 18, 2017 (CN) 201710842235.9

(51) Int. Cl. C12Q 1/686; C12Q 1/6851; B01L 3/00

(52) U.S. Cl. CPC C12Q 1/686; C12Q 1/6851; B01L 2300/0861; C12Q 2561/113; C12Q 2565/101; B01L 3/502769

(57) ABSTRACT

The invention relates to a nucleic acid determination method, which is implemented by the sealed reaction vessel including a tubular chamber and a channel connecting the tubular chamber; the first tubular chamber is provided with the sample and the first set reaction reagent, and the nth tubular chamber is provided with the nth set reaction reagent; the method comprises the following steps: sealing the reaction vessel, conducting the first set of reactions in the first tubular chamber, and the products in the first tubular chamber are transported to the latter tubular chamber through a channel; after the product of the former tubular chamber is transported to the nth tubular chamber, the nth set reaction is carried out in the nth tubular chamber. The nucleic acid determination method provided by the invention can ensure the sealed closed system and convenient and simple operation. Moreover, it does not need to use molecular probe, does not depend on the exonuclease activity of polymerase and is not limited by the length of target molecule.

### TECHNICAL FIELD

The invention relates to the field of molecular biology, in particular to a method for the qualitative or quantitative determination of nucleic acid.

### BACKGROUND OF THE INVENTION

With the development of biotechnology, modern molecular biology technology or genetic engineering technology is increasingly widely used in various biotechnology industries, especially in medical diagnosis. The application of these techniques often involves the qualitative and quantitative determination using molecular probes. The typical nucleic acid quantitative amplification reaction is polymerase chain reaction (PCR). For example, in the detection and diagnosis of RNA virus, it is necessary to purify the virus RNA first, then reverse transcription (RT) of RNA into cDNA, and then the quantitative amplification of cDNA (RT-PCR) is carried out. It can be seen that RT-PCR is a two-step reaction. The multi-step reaction also includes nested PCR. Nested PCR is to use an external primer to complete the first step of PCR, and then a pair of inner primers are used for the second reaction of products that obtains in the first step, and quantitative determination. Multiplex PCR is a reaction system (reaction tube) containing multiple pairs of primers. The multi-step reaction of nested PCR can be combined with multiple PCR, that is, the first step of nested PCR is to carry out multiple PCR, and the second step of nested PCR is to carry out multiple single quantitative PCR. Among them, quantitative PCR is to detect the total amount of products after each PCR cycle in the DNA amplification reaction, using external or internal controls as the standard. Through the analysis of PCR end products or the monitoring of PCR process, quantitative analysis of PCR initial template is carried out.

Nested PCR uses two or more pairs of PCR primers for amplification. The first pair of PCR primers are called outer primers (pairs) and the second pair are called inner primers (pairs). The second PCR amplification fragment is shorter than that of the first PCR product because of the inner primer binding to the first PCR product. The advantage of nested PCR is that if the first amplification produces an unintended fragment, the probability that second time the paired primers can also amplify the unintended fragment would be very low. Therefore, nested PCR has high specificity and sensitivity.

Multiplex PCR is a PCR reaction in which more than two pairs of primers are added to the same PCR reaction system and multiple nucleotide fragments are amplified at the same time. Multiplex PCR can analyse multiple genes in the same PCR reaction system, which has high efficiency.

RNA single strand molecules can be reverse transcribed into complementary DNA or cDNA molecules by reverse transcriptase (RT). cDNA can be used for PCR reaction as common DNA. This process is called RT-PCR.

The various type of PCR reactions described above can be carried out independently or in varied combinations. The existing technology often uses multiple reactors to carry out multi-step reactions respectively, which cannot meet the requirements of sealing, especially in the process of product transfer, the products are easy to cause contamination.

In addition, real-time quantitative nucleic acid amplification with fluorescence detection is commonly used in the existing technology for quantitative detection of nucleic acids, and fluorescent labeled molecular probes are required for the detection. The disadvantage of the existing method of quantitative probe is that the probe needs two or three tags, which is costly in design and manufacture. The method also requires the use of exonuclease activity of polymerase, which requires higher reagent requirements. The existing quantitative method of probe also requires that the target molecule has enough length, such as more than 50 base pairs. These requirements cannot be met in many applications.

In the existing technology, there are also methods for quantitative determination of nucleic acids using non-specific fluorescent dyes. This kind of fluorescent dye can bind with double stranded DNA and produce fluorescence. The signal generated is proportional to the amount of DNA, but it is not specific to the sequence of amplified DNA and thus cannot exclusively determine the specific DNA sequence.

### TECHNICAL ISSUE

In order to solve the above problems, the invention aims to provide a nucleic acid determination method which can ensure a closed system which is convenient and simple to operate.

Further, the purpose of the present invention is to provide a probe free method for qualitative or quantitative determination of nucleic acids that does not depend on the exonuclease activity of polymerase and is not limited by the length of the target molecule.

### TECHNICAL SOLUTIONS

To achieve the above purpose, the invention provides a nucleic acid determination method, which is implemented by a sealable reaction vessel that comprises at least two tubular chambers set up in sequence and at least one channel connecting at least two tubular chambers; wherein, the first tubular chamber is provided with the sample and first set reaction reagent; the nth tubular chamber is provided with the nth set reaction reagent, wherein n is equal to or greater than 2; the method comprises the following steps: sealing the reaction vessel, conducting the first set of reactions in the first tubular chamber, and the products in the first tubular chamber are transported to the latter tubular chamber through a channel; after the nth tubular chamber receives the product of the previous tubular chamber, the nth set reaction is carried out in the nth tubular chamber; then detects the product in the nth tubular chamber.

The invention adopts a sealed reaction vessel with at least two tubular chambers, and transported the product of the former tubular chamber to the latter through a channel connecting the tubular chambers. The product reacts as a substrate in the latter tubular chamber, and can realize multi-step PCR reaction, such as nested PCR, RT-PCR, multiplex PCR or two or more combinations of them.

In one embodiment, the first set reaction reagent includes the first pair of primers of nested PCR or RT-PCR or the first set of primers of multiplex PCR; the nth set reaction reagent includes the nth pair of primers of nested PCR or RT-PCR or multiplex PCR or the nth set of primers and affinity substances of multiplex PCR.

In each step of amplification, the products can be determined qualitatively and quantitatively by the detectable signal formed by the combination of affinity substances and products. Among them, affinity substances can be any one or more existing substances that can directly bind with products to form detectable signals. Different from the molecular probe, the affinity substance does not depend on the exonuclease activity of polymerase, so that any polymerase suitable for PCR can be used for the quantitative determination of the invention, thus conducive to the wider adaptation of quantitative determination, and does not need to special considerations for the molecular probe in the reaction design, and not limited by the length of the target molecule. The invention does not need to use the molecular probe with fluorescence mark for qualitative or quantitative determination, and effectively reduces the cost.

The first set reaction reagent and the nth set reaction reagent include not only corresponding primers and affinity substances that can be added as required, but also other reaction reagents known in the sector, such as polymerases, reverse transcriptases, nucleotides, buffers and other organic or inorganic substances. Reaction reagents and samples can be added into the corresponding tubular chamber by manual or automated methods prior to the reaction. The reaction reagent can also be added in advance, sealed, stored and transported, and the sample can be added to the corresponding tubular chamber during use. After the sample and reaction reagent are added, the reaction vessel can be sealed by physical, mechanical or chemical means for subsequent enzyme reaction.

According to the principle of nested PCR and RT-PCR, an external primer or a reverse transcription primer can be placed in the first tubular chamber, and a pair of internal primers can be placed in the second tubular chamber. If necessary, the third tubular chamber can be equipped with corresponding primers. In the quantitative reaction, the sample containing the target molecule is only added to the first reaction tube with external primers.

The principle of quantitative measurement in this method is that the time of signal generated in the second and subsequent tubular chambers is in a specific proportion to the number of sample target molecules in the first tubular chamber when the reaction is in progress. This is due to the fact that the product molecules in the former tubular chamber are transported to the latter in a limited process through such methods as molecular diffusion, liquid convection, and become the molecular substrate for the next reaction, the weight and speed of the transported product molecules are directly proportional to the product quantity of the previous reaction. The sensitivity of quantitative detection and the dynamic range of reaction can be adjusted by adjusting or controlling the transfer probability of molecules between reaction tubes.

Another embodiment of the invention includes temperature control for each tubular chamber, and the temperature control includes maintaining a constant temperature gradient in any tubular chamber or periodically changing the temperature in any tubular chamber.

The temperature of the reaction vessel can be controlled by all known methods. The temperature of the reactor can be in overall equilibrium, in overall change or temperature difference between different parts, especially each reaction chamber can maintain a temperature gradient. The temperature control method can be constant heating of the specific part of the tubular chamber, maintaining a constant temperature gradient in the tubular reactor, or temperature control with periodic change to make the tubular reactor produce a balanced periodic change of temperature. The effect is to make the molecules in the tubular chamber undergo different temperatures, so as to meet the requirements of different enzyme reaction conditions and achieve the purpose of nucleic acid amplification in the tubular chamber.

In some embodiments, the temperature control method includes synchronous temperature control for multiple tubular chambers.

During the quantitative determination of nucleic acid, the temperature of multiple tubular chambers shall be controlled synchronously, for example, the temperature of the second and the subsequent tubular chambers shall be controlled synchronously, so as to ensure that the second and the subsequent tubular chambers can react immediately at any time when they receive the products transported by the previous tubular chamber, and also ensure that the reaction conditions are consistent, and then quantitative analysis shall be conducted through signal detection.

An embodiment of the invention is that a medium is set up in the channel, In an embodiment of the invention, the products from the reaction in any tubular chamber can be transported to the latter through liquid convection or molecular diffusion in the channel.

The medium in the channel may be any medium capable of transporting the product, for example, a liquid. After the reaction in the first tubular chamber, the product molecules can be transported to another tubular reactor through the medium. The mode of transport can be liquid convection or molecular diffusion, so that part of the product molecules is transported from one tubular chamber to the next. The amount, speed and time of transport are determined by the sample target molecular weight in the previous reaction tube. It can be used as the basis of quantitative measurement.

In some embodiments, the channel is set up at the upper end of the tubular chamber.

The connecting part of the channel and the tubular chamber can be the upper end, the middle end or the lower end of the tubular chamber. The preferred configuration is that the channel connects multiple tubular chambers at the upper end of the tubular chamber. This connection is conducive to prevent the mixing of substances between different tubular chambers.

An embodiment of the invention is that the tubular chamber is cylindrical or conical, the inner diameter is between 0.1 mm and 10 mm, and the chamber wall thickness is between 0.05 and 5 mm; the ratio of the depth of the tubular chamber to the inner diameter is greater than or equal to 2.

The adoption of cylindrical or conical tubular chamber is beneficial to keep the temperature of the tubular chamber uniform or to form a constant temperature gradient. The inner diameter, wall thickness and the ratio of the thickness to the inner diameter of the tubular chamber can meet the general reaction needs and maintain safety and stability, as well as saving costs.

In some embodiments, the reaction vessel further comprises a raised arc-shaped connecting part which is located between adjacent tubular chambers in the channel.

The adjacent tubular chambers are connected by arc connecting parts, which can avoid the formation of dead space and make the transportation of the product molecules between different tubular chambers more smoothly in the medium of the channel.

In some embodiments, the affinity substance includes dyes.

Affinity substance can be any existing substance that can bind with nucleic acid products to generate a detectable signal, such as dye, especially fluorescent dye, which can bind with nucleic acid products to form fluorescent signal. Affinity substances can also be some surfactants, metal complexes, proteins and other substances that can bind with the products.

In some embodiments, the detectable signal includes the optical signal or the electrical signal. Further, the optical signal includes fluorescence signal, light absorption signal, infrared absorption signal, Raman scattering signal or chemiluminescence signal.

Through the combination of different affinity substances and products, optical or electrical signals can be generated. For the transparent reaction vessel, it is advantageous to carry out qualitative or quantitative determination by optical signal, and it also can carry out real-time quantitative determination. The optical signal can be detected by existing optical signal detection instruments.

### BENEFICIAL EFFECTS

The nucleic acid determination method of the invention does not need a probe, does not depend on the exonuclease activity of polymerase, is not limited by the length of the target molecule, ensures a closed reaction system, and is convenient and simple in operation.

### DESCRIPTION OF FIGURES

FIG. 1 is a structural diagram of a reaction vessel used in an example of the determination method of the present invention.

FIG. 2 is a structural diagram of a sealed reaction vessel used in an example of the determination method of the present invention.

FIG. 3 is a schematic diagram of the reaction process used in an example of the determination method of the present invention.

FIG. 4 is a result analysis diagram used in an example of the determination method of the present invention.

### EXAMPLE OF THE INVENTION

The nucleic acid determination method of the invention will be further described with the attached figures and examples.

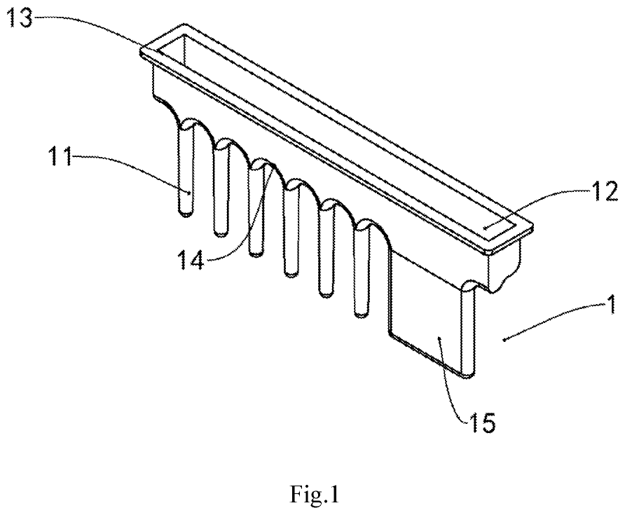

The nucleic acid test method of the example is implemented through the sealable reaction vessel. One of the sealable reaction vessels is shown in FIG. 1. The reaction vessel 1 includes at least two tubular chambers 11 set up in sequence. It may be sequentially named the first tubular chamber to the nth tubular chamber, where n is equal to or greater than 2.

Different tubular chambers 11 can carry out different reactions in multi-step reactions, and the number of tubular chambers 11 can be determined according to the actual needs of the reaction. The tubular chamber 11 may be connected by one or more channels 12 at the upper, middle or lower ends of the tubular chamber 11, and the channels 12 may enable the reaction products to be transported between the tubular chambers 11 under sealed conditions, so as to enable the multi-step enzyme reaction. Specifically, in this example, tubular chambers 11 are connected by the channel 12 at the upper end of the tubular chamber 11, which is conducive to preventing unnecessary mixing of reaction materials between different tubular chambers 11. Channel 12 can be designed into different shapes as required, such as tubular, channel, etc.

In this example, the inner diameter of the tubular chamber 11 is between 0.1 mm and 10 mm, and the thickness of the tubular chamber wall is between 0.05 and 5 mm; the ratio of the depth of the tubular chamber 11 to the inner diameter is greater than or equal to 2. When the dimension of the tubular chamber 11 is within the above range, it can meet the needs of general multi-step enzyme reaction, and it is safe and stable, saving cost.

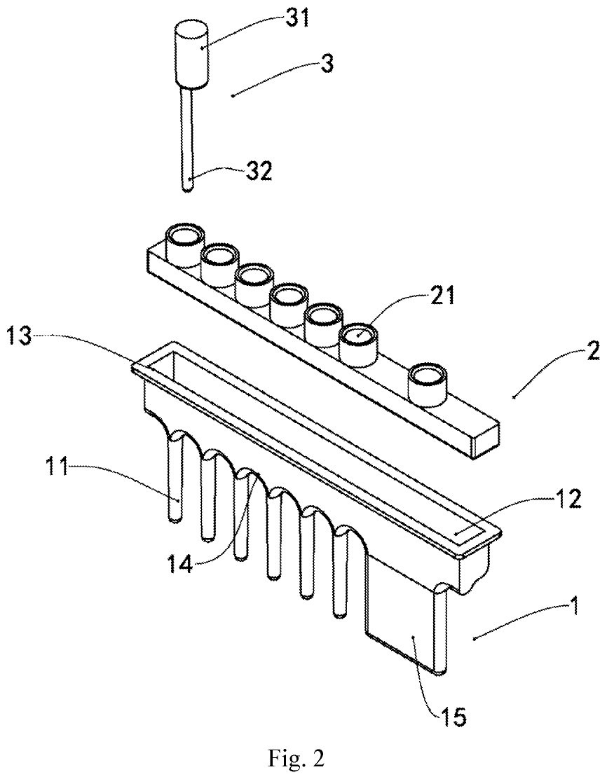

The Reaction Vessel 1 may also include an Opening 13, preferably located at the upper end of the Channel 12. The Opening 13 can be sealed in any of the existing ways. As shown in FIG. 2, in this example, the Opening 13 can be sealed and worked with the Cover 2, which is provided with a Through Hole 21 for adding samples and sampling. Seal 3 can be inserted in Through Hole 21. Each Seal 3 includes Sealing Plug 31 which can be worked with Through Hole 21, and Sealing Rod 32 which can be worked with at least a part of the Tubular Chamber 11 is fixedly or movably connected at the lower end of the Sealing Plug 31 according to the need, and the Sealing Rod 32 can also be used for sample collection before reaction.

At least two adjacent Tubular Chambers 11 are provided with raised Arc-Shaped Connecting Part 14 in the Channel 12. The Arc-Shaped Connecting Part 14 can avoid dead space, which is conducive to the transmission of products between adjacent Tubular Chambers 11. In addition, in order to facilitate the placement of the reaction reagent or sample, the Storage Chamber 15 can also be set up in the Reaction Vessel 1.

Preferably, the Reaction Vessel 1 is made of transparent materials such as plastic, glass, etc., so it has a transparent outer surface, and the reaction result can be continuously and rapidly detected by optical method in real time. Reaction Vessel 1 can be formed by machining or injection moulding.

When the above Reaction Vessel 1 is applied to nucleic acid detection, the sample and the first set reaction reagent need to be placed in the first tubular chamber, and the nth set reaction reagent needs to be placed in the nth tubular chamber, where n is one or more positive integers starting from 2. According to the principle of nested PCR, the first reaction reagent includes not only the components known in the sector such as polymerase, nucleotide and buffer, but also the first pair of primers of nested PCR, i.e. external primers. In addition to the components known in the sector such as polymerases, nucleotides and buffers, the nth reaction reagent also includes the nth pair of primers of nested PCR, i.e. inner primers, and affinity substances. In the quantitative reaction, the sample containing the target molecule is only added to the first reaction tube with external primers.

The reagent can also be stored in the Tubular Chamber 11 in advance, and then stored and transported after being sealed. Only the sample needs to be added during the use. The Tubular Chamber 11 can also be filled with reaction reagents and biological samples by manual or automated method before use.

Affinity substances can be any existing substance that can bind with nucleic acid products to generate detectable signal, such as dye, especially fluorescent dye, which can bind with nucleic acid products to generate fluorescent signal. Affinity substances can also be some surfactants, metal complexes, proteins and other substances that can bind with products and produce detectable signals.

Through the combination of different affinity substances and products, optical or electrical signals can be generated. For the transparent reaction vessel, it is advantageous to measure qualitatively or quantitatively by optical signal, which includes fluorescence signal, light absorption signal, infrared absorption signal, Raman scattering signal or chemiluminescence signal, and can be detected in real time.

One or more media are set up in the Channel 12, the media can be liquid, and the products in the Tubular Chamber 11 are transported to the latter through the media. The mode of transport can be liquid thermal convection or molecular diffusion.

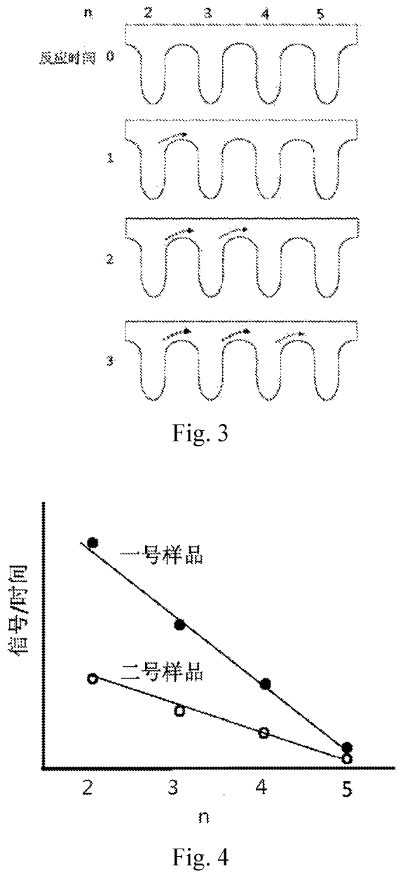

In the determination of nucleic acid, the Reaction Vessel 1 is sealed first to avoid contamination of the product. Then the temperature of each tube chamber is controlled for nested PCR. Specifically, the first amplification reaction of nested PCR is carried out in the first tubular chamber, the first amplification reaction can be carried out independently, and then the product obtained is transported to the second tubular chamber (n=2). As shown in FIG. 3, the second amplification reaction of nested PCR is carried out in the second tubular chamber (n=2). As the reaction proceeds, the amount of the product increases gradually, and the product can also be automatically transported to the third tubular chamber (n=3) through the channel gradually. The next amplification reaction continues, and until the required multi-step reaction is completed. The products in the nth tubular chamber bind with affinity substances to produce detectable signals. From the second tube chamber to the fifth tube chamber, the detectable signal can be generated step by step, and the detectable signal increases with time. The time in FIG. 3 starts from the transportation of products in the first tubular chamber.

Temperature control is needed for enzymatic reactions. Generally, the enzyme reaction is between 15° C. and 99° C. Currently known methods can be used to control the temperature of biological enzyme reaction in tubular reactor, such as infrared light, hot/cold air, cold/hot solid or liquid substances, electromagnetic induction, etc. The sealed reaction device can be inserted into the temperature control instrument for temperature control. According to the requirements of the reaction, any Tubular Chamber 11 can withstand constant temperature or temperature with periodic change, and there can also be temperature in equilibrium or temperature in a gradient inside the tubular chamber 11. For example, similar to the traditional PCR temperature control method, the temperature of the temperature control instrument changes periodically under the control of a computer program. For example, the temperature of the temperature control instrument is kept for a few seconds to a few minutes under a certain temperature, and the Tubular Chamber 11 is completely inserted into the heating part of the temperature control instrument. In this process, the liquid temperature in the Tubular Chamber 11 is nearly uniform. For example, in the gradient temperature control method with constant temperature heating, the temperature of the temperature control instrument remains unchanged under the control of a computer program, and only part of the Tubular Chamber 11 contacts the heating part of the temperature control instrument. When the bottom is heated, the bottom temperature will be higher than the top temperature, and the liquid in the Tubular Chamber 11 will have a temperature gradient. Due to the relatively high density or specific gravity of the liquid with low temperature in the upper part, the liquid in the upper part and the liquid in the lower part will produce convection. The effect is to drive the molecular flow in the tubular chamber and endure different temperatures, so as to meet the requirements of different enzyme reaction conditions and achieve the purpose of DNA amplification in the Tubular Chamber 11. The tubular structure of the tubular Chamber 11 brings more flexibility to the instrument design.

In the quantitative determination of nucleic acid, the temperature of the tubular chamber shall be controlled synchronously to ensure that the second and the subsequent tubular chambers can react immediately when receiving the products transported by the previous tubular chamber at any time, so as to ensure that the reaction conditions are consistent.

The principle of quantitative measurement with this method is that, when the reaction is going on, the product molecules in the former tubular chamber will be transported to the latter tubular chamber through channels, such as diffusion, etc., and become the molecular substrate of the latter reaction. The quantity and speed of the product molecules transported are in direct proportion to the product quantity of the previous reaction. The sensitivity of quantitative detection and the dynamic range of reaction can be adjusted by adjusting or controlling the transfer probability of molecules between reaction tubes.

As shown in FIG. 4, in the tubular chambers with consecutive reactions, the ratio of signal to time is proportional to the position of the tubular chamber, wherein the ratio of signal to time refers to that when the signal reaches the unsaturated state, i.e. linear growth period, which is expressed in logarithmic form. When the gradient of sample 1 is greater than that of sample 2, the number of target molecules of sample 1 is greater than that of sample 2.

The method of the example can be applied to qualitative and quantitative tests of various nucleic acids.

For example, the method can be used for the detection of human EGFR gene mutations. Specifically, it includes the following steps:

In the first tube chamber, the outside set reaction of nested PCR is carried out. The mutation fragment of EGFR tyrosine kinase domain (TM domain) is amplified by DNA polymerase and an external primer. Specifically, the exon 18-22 sequence covering multiple mutation sequences is amplified by outside primer pair.

DNA template, 200 μM dNTPs, 20 mm Tris HCl, 10 mm (NH₄)₂SO₄, 2 mm MgSO₄, 0.1% Triton®-X-100, 1 unit DNA polymerase and 0.3 μM external primer pairs are added in the first tubular chamber of 50 uL reaction volume.

EGFR OutF1:
5′-CAATG CCATC CACTT GATAG G-3′
EGFR OutR1:
5′-GATCG GCCTC TTCAT GCGAA GG-3′

Then, in the first chamber, DNA amplification is carried out by constant temperature heat convection in the same tube. The same tube constant temperature convection reaction is carried out for 15 minutes.

(2) The products of the first round of amplification reaction are transported to the second tubular chamber through channels, and the products in the former tubular chamber are successively transported to the latter. Each successive tubular chamber contains a specific primer pair for mutation gene test (take T790r and L858R mutations as examples) and the affinity substance for detection:

T790 wild-type detection is carried out in the second tubular chamber, which contained 200 μM dNTPs, 20 mM Tris-HCl, 10 mM (NH₄)₂SO₄, 10 mM KCl, 2 mM MgSO₄, 0.1% Triton®-X-100, 0.25×Cyber Green dye (AAT Bioquest, USA) and 1 unit DNA polymerase, and 0.3 μM inner primer set in the nested PCR:

EGFR T790F:
5′-ACCTC CACCG TGCAG CTCAT CAC-3′
EGFR 790R:
5′-GTGTT CCCGG ACATA GTCCA -3′.

In the third chamber, there are 200 μM dNTPs, 20 mM Tris HCl, 10 mM (NH₄)₂SO₄, 10 mM KCl, 2 mM MgSO₄, 0.1% Triton®-X-100, 0.25×Cyber Green Dye (AAT Bioquest, USA) and 1 unit DNA polymerase, and 0.3 μM inner primer set in the nested PCR:

EGFR M790F:
5′-ACCTC CACCG TGCAG CTCAT CAT-3′
EGFR 790R:
5′-GTGTT CCCGG ACATA GTCCA -3′.

L858 wild-type detection is carried out in the fourth tubular chamber, the fourth tubular chamber comprised of 200 μM dNTPs, 20 mM Tris HCl, 10 mM (NH₄)₂SO₄, 10 mM KCl, 2 mM MgSO₄, 0.1% Triton®-X-100, 0.25×Cyber Green Dye (AAT Bioquest, USA) and 1 unit DNA polymerase, as well as 0.3 μM inner primer set in the nested PCR:

EGFR L858F:
5′-GTCAA GATCA CAGAT TTTGG GCT-3′
EGFR OutR1:
5′-GATCG GCCTC TTCAT GCGAA GG -3′.

The fifth tubular chamber comprised of 200 μM dNTPs, 20 mM Tris HCl, 10 mM (NH₄)₂SO₄, 10 mM KCl, 2 mM MgSO₄, 0.1% Triton®-X-100, 0.25×Cyber Green Dye (AAT Bioquest, USA) and 1 unit DNA polymerase, and 0.3 μM inner primer set in the nested PCR:

EGFR R858F:
5′-GTCAA GATCA CAGAT TTTGG GCG-3′
EGFR OutR1:
5′-GATCG GCCTC TTCAT GCGAA GG-3′.

From the second tube chamber to the fifth tube chamber, the constant temperature convection reaction is carried out simultaneously for 15 minutes.

(3) The signal from the second tube chamber to the fifth tube chamber can be detected continuously, and the target molecule number of the sample can be determined quantitatively. The genotypes of the samples are determined by comparing wild type and mutant type.

Finally, it should be emphasized that the above is only a preferred example of the invention and is not used to limit the invention. For those skilled in the sector, the invention may have various changes. Any modification, equivalent replacement and improvement made within the spirit and principles of the invention shall be included in the protection scope of the invention.

### INDUSTRIAL APPLICABILITY

It can be seen from the above that the nucleic acid determination method provided by the invention combines a sealed reaction vessel with tubular chambers and channels with a nested PCR reaction for qualitative or quantitative determination, which can prevent the product from causing contamination, and the reaction has high sensitivity and accuracy, does not need to use molecular probes, does not rely on the exonuclease activity of polymerase and is not limited by the length of the target molecule. The nucleic acid determination method of the invention can realize multi-step PCR reaction, such as nested PCR, RT-PCR, multiplex PCR or two or more combinations of them.

1-10. (canceled)

11. A nucleic acid determination method comprising:
(i) assembling a reaction vessel, wherein the reaction vessel comprises (a) plurality of a tubular chamber, wherein a first tubular chamber and a second tubular chamber is set up in sequence (b) a channel connecting with the first tubular chamber and the second tubular chamber;
(ii) providing a sample in the first tubular chamber, the tubular chamber is provided with a reaction reagent;
(iii) sealing the reaction vessel;
(iv) conducting a first group set of reaction in the first tubular chamber to form a first product;
(v) transporting the first product to the second tubular chamber through the channel sequentially;
(vi) conducting a second group set of reaction in the second tubular chamber to form a second product; and
(vii) detecting the second product in the second tubular chamber by a detectable signal.

12. The nucleic acid determination method of claim 11, wherein the tubular chamber comprises (i) the reaction reagent comprising: a first pair of primers for a nested PCR or a reverse transcription reaction primer for RT-PCR or a first set of primers for a multiplex PCR, a nth pair of primers, and (ii) an affinity substance for nested PCR or RT-PCR or multiplex PCR or combination thereof.

13. The nucleic acid determination method of claim 11, wherein the tubular chamber comprises a temperature control to either maintain a constant temperature gradient in the tubular chamber or periodically change an overall temperature of the tubular chamber.

14. The nucleic acid determination method of claim 13, wherein the temperature control comprises a synchronous temperature control of the tubular chamber.

15. The nucleic acid determination method of claim 11, wherein the channel comprises a media.

16. The nucleic acid determination method of claim 11, wherein the first product of the first tubular chamber is transported to the second tubular chamber through fluid convection or molecular diffusion in the channel.

17. The nucleic acid determination method of claim 11, wherein the tubular chamber is cylindrical or tapered conical, an inner diameter is between 0.1 mm and 10 mm, and a pipe tubular chamber wall thickness is between 0.05 and 5 mm; a ratio of depth of the tubular chamber to the inner diameter is greater than or equal to 2.

18. The nucleic acid determination method of claim 11, wherein the reaction vessel further comprises a raised arc-shaped connecting part located between the first tubular chamber and second tubular chamber in the channel.

19. The nucleic acid determination method of claim 12, wherein the affinity substance comprises a substance with an ability to interact with a nucleic acid product of the tubular chamber; wherein the substance comprises a dye, a surfactant, a metal complex, a protein or a combination thereof.

20. The nucleic acid determination method of claim 11, wherein the detectable signal is an optical signal, an electrical signal or combination thereof; the optical signal is a fluorescence signal, a light absorption signal, an infrared light absorption signal, a Raman scattering signal or a chemiluminescence signal or combination thereof.

21. A multi-step reaction vessel comprising:
(a) plurality of a tubular chamber, wherein a first tubular chamber and a second tubular chamber set up in sequence;
(b) a channel connecting the first tubular chamber and the second tubular chamber; wherein the channel transports a first product of the first tubular chamber to the second tubular chamber sequentially.

22. The multi-step reaction vessel of claim 21, wherein the tubular chamber comprises a temperature control; the channel comprises an opening; wherein the opening is sealed by a first seal comprising a cover.

23. The multi-step reaction vessel of claim 22, wherein the cover comprises a through-hole; wherein the through-hole is configured with a seal comprising a sealing plug and a sealing rod.

24. The multi-step reaction vessel of claim 23, wherein the sealing rod configured with the tubular chamber is either fixed or movably connected to a lower end of the sealing plug.

25. The multi-step reaction vessel of claim 21, further comprising a storage chamber.

26. The multi-step reaction vessel of claim 21, wherein the second tubular chamber comprises an affinity substance comprising a substance with an ability to interact with a nucleic acid product; wherein the substance comprises a a dye, a surfactant, a metal complex, a protein or a combination thereof.

27. The multi-step reaction vessel of claim 21, wherein the channel comprises a media.

28. The multi-step reaction vessel of claim 21, wherein the first tubular chamber and the second tubular chamber is connected with a part to avoid formation of a dead space between the first tubular chamber and the second tubular chamber; wherein the part comprises an arc shaped part.

29. A nucleic acid determination method in a closed system comprising:
(i) sealing a reaction vessel comprising at least two tubular chamber comprising a first tubular chamber and second tubular chamber, the tubular chamber comprises a reaction reagent, the first tubular chamber comprises a sample, second tubular chamber comprises an affinity substance;
(ii) conducting a first group of reactions in the first tubular chamber to form a first product;
(iii) transporting the first product to the second tubular chamber through a channel to form a second product;
(iv) detection of the second product in the second tubular chamber.

30. The nucleic acid determination method of claim 29, wherein the method is a probe free method for a qualitative or a quantitative determination of nucleic acid.
